(19) United States
(12) Patent Application Publication
(10) Pub. No.: US 2006/0121598 A1
(43) Pub. Date: Jun. 8, 2006

(54) System for detecting at least one chemical substance

(76) Inventors: Guillaume Herlem, Besancon (FR); Tijani Gharbi, Besancon (FR); Philippe, Gerard, Lucien Humbert, Ornans (FR)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 10/521,681

(22) PCT Filed: Jul. 10, 2003
(86) PCT No.: PCT/FR03/02169

§ 371 Date: Jul. 1, 2005

(30) Foreign Application Priority Data
Jul. 18, 2002 (FR) FRN 02 09219

(51) Int. Cl. C12M 1/34

(52) U.S. Cl. 435/287.1; 435/287.9

(57) ABSTRACT

A detector system for detecting a chemical substance, the system being of the type comprising a measurement sensor for selectively sensing the chemical substance to be detected, and a measurement unit associated with the measurement sensor for the purpose of being connected to a processor unit to determine whether said chemical substance for detection is present or absent. The measurement sensor comprises an olfactory neuron selected for selectively sensing the chemical substance to be detected, and the olfactory neuron is secured to a support in order to co-operate with the measurement unit.

The present invention relates to systems for detecting at least one chemical substance.

More particularly, amongst such detector systems, the invention relates to those which comprise a measurement sensor for selectively sensing said at least one chemical substance to be detected, and a measurement unit associated with the measurement sensor and designed to be connected to a processor unit to determine whether or not the chemical substance for detection is present.

In those known types of detector system, the measurement sensor is generally constituted by a pair of electrodes placed in a fluid to be analyzed, the pair of electrodes being designed to detect the presence or the absence in the fluid to be analyzed of a chemical substance such as a specific molecule.

The pair of electrodes is generally constituted by a reference electrode and a measurement electrode having a conductive polymer coating applied thereto. The conductive polymer coating is selected to generate an intrinsic electrical signal when a determined molecule is selectively absorbed at the surface of said polymer coating. Nevertheless, such detector systems present sensitivity that is relatively low, since polymer coatings are not adapted to detecting very low concentrations of a specific molecule.

Furthermore, polymer coatings are adapted solely for detecting specific molecules presenting a chemical formula that is very simple. Thus, in order to detect complex molecules, it is essential to implement a series of a plurality of measurement electrodes carrying distinct polymer coatings in the hope of being able to detect molecules of complex chemical formula. In addition, polymer coatings tend to saturate quickly in the presence of a fairly high concentration of the molecules to be detected, which means that it is not possible to evaluate the concentration of said target molecules accurately.

A particular object of the present invention is to mitigate the above-mentioned drawbacks.

To this end, the invention provides a detector system for detecting at least one chemical substance, which system is characterized in that the measurement sensor comprises at least one olfactory neuron selected for selectively sensing the chemical substance to be detected, and in that the olfactory neuron is secured to a support in order to co-operate with the measurement unit.

In preferred embodiments of the invention, recourse may optionally also be had to one or more of the following dispositions:

the support is covered at least in part in electrical insulation on which the olfactory neuron is secured;

the electrical insulation comprises a polymer also suitable for securing the olfactory neuron;

the polymer is deposited on the support electrochemically by dipping a reference electrode and at least a portion of the support in a liquid electrolyte based at least on a salt and a solvent, and by bringing the support and the reference electrode to a potential that is not less than the oxidation potential of said solvent;

the solvent is selected from a pure saturated aliphatic primary diamine, a pure saturated aliphatic primary triamine, a saturated aliphatic amino-thiol, and a saturated aliphatic dithiol;

the measurement unit comprises at least one measurement electrode and a reference electrode in contact with the olfactory neuron, said measurement and reference electrodes being for connection to the processor unit;

the olfactory neuron presents a cell body which is extended on one side by dendrites and on an opposite side by an axon presenting a plasma membrane, and the measurement electrode is disposed inside the plasma membrane of the axon, while the reference electrode is placed in contact with the surface of the plasma membrane of said axon; and

the measurement unit comprises firstly emitter means for emitting excitation light towards the olfactory neuron to enable the excitation light to interact with the chemical substance to be detected in order to produce radiation for detection, and secondly reception means for receiving the radiation for detection as emitted by the chemical substance, said reception means being connected to the processor unit.

Other characteristics and advantages of the invention appear from the following description of three embodiments thereof given as non-limiting examples and with reference to the accompanying drawings.

In the drawings:

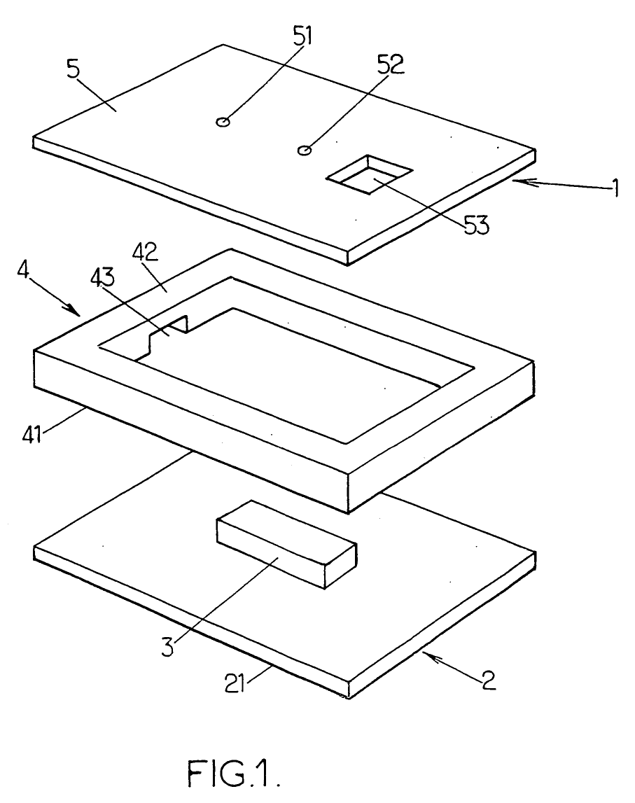

FIG. 1 is a diagrammatic exploded perspective view of a portion of a detector system of the invention;

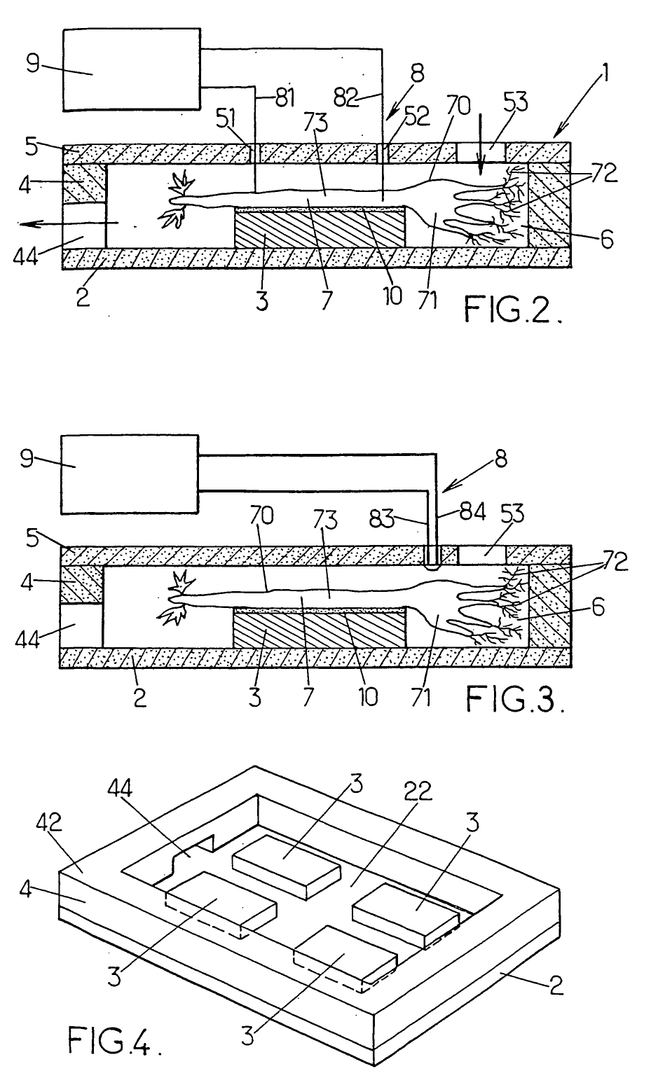

FIG. 2 is a section view of a detector system constituting a first embodiment of the invention;

FIG. 3 is a section view of a detector system constituting a second embodiment of the invention; and

FIG. 4 is a diagrammatic perspective view of a portion of a detector system constituting a third embodiment.

In the various figures, the same references are used to designate elements that are identical or similar.

FIGS. 1 and 2 show a first embodiment of the detector system in accordance with the invention. The detector system comprises a base 2 in the form of a rectangular plate having a bottom face 21 that is to rest on any suitable support, and a top face 22 that is opposite from and parallel to the bottom face 21, and serving to receive a support 3. As described below, the support 3 serves to enable the measurement sensor to be secured.

The detector system also comprises a spacer 4 which in the example described herein is in the general form of a rectangular frame whose outline is substantially equal to the outline of the base 2. The spacer 4 likewise comprises a bottom face 41 for securing to the top face 22 of the base 2, and a top face 42 that is to have a cover 5 secured thereto.

The cover 5 is likewise in the form of a rectangular plate whose outline is substantially identical to the outline of the spacer 4 and of the base 2.

Thus, as can be seen in FIG. 2, when the spacer 4 is secured to the base 2 and the cover 5 is resting on the top face 42 of the spacer 4, the detector system presents an enclosure 6 in which the support 3 is situated. The support 3 is adapted to have an olfactory neuron 7 secured thereto which is selected for its ability to sense or trap selectively a specific and predetermined chemical substance present in a fluid for analysis that is placed in the enclosure 6 of the detector system.

The olfactory neuron which is covered in a plasma membrane 70 comprises a central portion 71, or neuron cell body, which is extended at one end by dendrites 72 formed by a plurality of receptor cilia, and at its opposite end by an axon 73 which extends to an axon termination.

In general, the olfactory neuron devotes a fraction of its genes to controlling the synthesis of large molecules, known as receptor proteins, that the neuron places in the membrane of the cilia or dendrites 72. These molecular receptors are quite distinct depending on which olfactory neuron has been selected, and they can fix or bind and also recognize a large number of chemical substances or odor molecules. The olfactory neuron for placing on the support 3 is selected from a plurality of olfactory neurons as a function of its properties, or more exactly of the receptor properties of the cilia for sensing or trapping a particular odor molecule.

By way of example, if the detector system is to sense certain odor molecules present in a polluted liquid such as water, the olfactory neuron may be extracted from the olfactory neuron system of a trout, which system includes certain olfactory neurons that are adapted to recognize very low concentrations of very specific odor molecules.

In order to secure the selected olfactory neuron 7 on the support 3, a polymer is used having electrical insulation properties while also serving to secure the olfactory neuron.

The polymer 10 is deposited on the support 3 electrochemically by dipping a reference electrode (not shown) and at least a portion of the support 3 in a liquid electrolyte based on at least one salt and a solvent. The support 3 and the reference electrode may be made of platinum, of gold, or of vitreous carbon, or it may be based on p-type silicon of controlled thickness. Thereafter, the support 3 and the reference electrode are taken to a potential that is not less than the oxidation potential of the solvent so as to enable the polymer 10 to become fixed on the support 3 and on the reference electrode. By way of example, the solvent used may be selected from a pure saturated aliphatic primary diamine, a pure saturated aliphatic primary triamine, or a saturated aliphatic amino-thiol, or indeed a saturated aliphatic dithiol.

Thereafter, it suffices to remove the support 3 from the electrolyte and to place the olfactory neuron selected for selectively sensing an odor molecule on the polymer 10 as obtained in this way and that is covering the support. The polymer presents the property of being an electrically insulating polymer, thus making it possible, while it is being made by electrosynthesis, to ensure that formation thereof stops naturally, thereby obtaining a polymer coating that is very thin. In addition, the polymer is advantageously selected to be non-toxic and biocompatible with the selected olfactory neuron.

In the first embodiment shown in FIG. 2, the olfactory neuron 7 is in contact with a measurement system 8, itself connected to a processor unit 9 adapted to analyze the information as obtained directly by the measurement unit 8. The measurement unit comprises a first reference microelectrode 81 presenting one end connected to the processor unit 9 and a second end placed on the surface of and in contact with the plasma membrane 70 of the axon 73 of the olfactory neuron 7, and a second measurement microelectrode 82 presenting a first end connected to the processor unit 9 and a second end placed inside the plasma membrane 70 of the axon 73 of the olfactory neuron 7.

The cover 5 of the detector system has two through openings 51 and 52 for receiving the microelectrodes 81 and 82 in sealed manner. The cover 5 also has a through window 53 for establishing communication between the external medium and the enclosure 6. The spacer 4 may also have a recess 43 in its bottom face 41 so as to co-operate with the top face 22 of the base 2 to define a window 44. By way of example, the through window 53 in the cover 5 may constitute an inlet orifice for the fluid to be analyzed within the enclosure 6, while the opening 44 constitutes an exhaust orifice for the fluid to be analyzed in the enclosure 6 after it has come into contact with the olfactory neuron 7, and more exactly with the dendrites 72 of the olfactory neuron.

While the detector system is not being used for the purpose of analyzing a fluid, the enclosure 6 is filled with a specific culture medium enabling the olfactory neuron to be kept alive, with the inlet and outlet orifices 53 and 43 then being temporarily closed by appropriate means such as plugs.

Conversely, while a fluid is being analyzed, or more exactly while the presence or absence of a specific molecule is being detected in the fluid, it suffices to replace the culture medium that is inside the enclosure 6 for the purpose of keeping the olfactory neuron 7 alive with the fluid that is to be analyzed.

When the fluid for analysis is present inside the enclosure 6, the olfactory neuron that has been selected for selectively sensing a specific odor molecule then fixes said odor molecules by means of its dendrites 72. This capture of the specific odor molecule then modifies the concentration of electrically charged molecules such as potassium ions K⁺ and sodium ions Na⁺ along the axon 73 of the neuron 7, thereby changing its intrinsic electrical resistance. This change in the intrinsic electrical resistance of the olfactory neuron 7 is then detected by means of the two microelectrodes 81 and 82, thus enabling the processor unit 9 to analyze and interpret the measurements taken in order to issue information concerning the presence and the concentration of the specific odor molecule present in the analyzed fluid. The measurement and reference electrodes 82 and 81 are disposed respectively inside the plasma membrane 70 and on the surface of the plasma membrane 70 of the axon 73, thus making it possible, by way of example, to measure the Na⁺ ion current that enters into the plasma membrane 70 through the voltage-dependent Na⁺ channels or the K⁺ ion current that passes through the plasma membrane 70 towards the enclosure 6 via voltage-dependent K⁺ channels. This electrophysiological technique making use of the properties of the K⁺ and Na⁺ ion channels of neurons in general is also known as the “patch-clamp” technique.

In a second embodiment of the invention as shown in FIG. 3, the measurement unit 8 comprises an excitation optical fiber 83 which has a first end connected to one or more light sources integrated in the processor unit 9, and a second end housed in sealed manner in a through opening made in the cover 5. This excitation optical fiber 83 serves to emit excitation light towards the olfactory neuron 7, and more exactly towards the cell body 71, so as to enable the excitation light to interact with the odor molecule(s) trapped in the olfactory neuron. By way of example, the excitation light may be selected to interact by the phenomenon of fluorescence with the Ca²⁺ ions released by the soma or cell body 71 of the olfactory neuron 7. The fluorescence radiation as emitted in this way from the cell body 71 of the olfactory neuron 7 is received by a reception second optical fiber 84 which delivers the radiation for detection to the processor unit 9. Naturally, the processor unit 9 and the light supply can be adapted depending on the optical fiber(s) used and on the phenomena that are to be studied such as phenomena of absorption, fluorescence, resonance, or interferometric phenomena depending on the way in which the detector optical fibers 82 and 84 are made. In addition, the ends of the optical fibers 83, 84 placed in the opening made in the cover 5 can be associated with an optical system 85 for focusing the excitation light on the olfactory neuron 7 and also for optimizing reception of the radiation to be detected by the reception optical fiber 84. For example, the Ca²⁺ ion flux can be measured by quantifying the fluorescence of a substance such as rhodamine B. The fluorescence of rhodamine B is blocked by the concentration of the Ca²⁺ ion.

As can be seen in FIG. 4 which shows a third embodiment of the invention, the enclosure 6, or more exactly the base 2 may be provided with a plurality of supports 3, each support 3 serving to have secured thereon a distinct olfactory neuron enabling each to detect a specific odor molecule, if present. Under such circumstances, each olfactory neuron secured to a support 3 is associated with a measurement unit 8 connected to a processor unit 9. Furthermore, the detector system is of very small dimensions, the base 2, the spacer 4, and the cover 5 being suitable for being made using pieces of silicon shaped by chemical machining.

1. The detector system for detecting at least one chemical substance, the system being of the type comprising a measurement sensor for selectively sensing the chemical substance to be detected, and a measurement unit associated with the measurement sensor for the purpose of being connected to a processor unit to determine whether said chemical substance for detection is present or absent, the system being characterized in that the measurement system comprises at least one olfactory neuron selected for selectively sensing the chemical substance to be detected, and in that the olfactory neuron is secured to a support in order to co-operate with the measurement unit.

2. The detector system according to claim 1, in which the support is covered at least in part in electrical insulation on which the olfactory neuron is secured.

3. The detector system according to claim 2, in which the electrical insulation comprises a polymer also suitable for securing the olfactory neuron.

4. The detector system according to claim 3, in which the polymer is deposited on the support electrochemically by dipping a reference electrode and at least a portion of the support in a liquid electrolyte based at least on a salt and a solvent, and by bringing the support and the reference electrode to a potential that is not less than the oxidation potential of said solvent.

5. The detector system according to claim 4, in which the solvent is selected from a pure saturated aliphatic primary diamine, a pure saturated aliphatic primary triamine, a saturated aliphatic amino-thiol, and a saturated aliphatic dithiol.

6. The detector system according to claim 1, in which the measurement unit comprises at least one measurement electrode and a reference electrode in contact with the olfactory neuron, said measurement and reference electrodes being for connection to the processor unit.

7. The detector system according to claim 6, in which the olfactory neuron presents a cell body which is extended on one side by dendrites and on an opposite side by an axon presenting a plasma membrane, and the measurement electrode is disposed inside the plasma membrane of the axon, while the reference electrode is placed in contact with the surface of the plasma membrane of said axon.

8. The detector system according to claim 1, in which the measurement unit comprises firstly emitter means for emitting excitation light towards the olfactory neuron to enable the excitation light to interact with the chemical substance to be detected in order to produce radiation for detection, and secondly reception means for receiving the radiation for detection as emitted by the chemical substance, said reception means being connected to the processor unit.

9. The detector system according to claim 2, in which the measurement unit comprises firstly emitter means for emitting excitation light towards the olfactory neuron to enable the excitation light to interact with the chemical substance to be detected in order to produce radiation for detection, and secondly reception means for receiving the radiation for detection as emitted by the chemical substance, said reception means being connected to the processor unit.

10. The detector system according to claim 3, in which the measurement unit comprises firstly emitter means for emitting excitation light towards the olfactory neuron to enable the excitation light to interact with the chemical substance to be detected in order to produce radiation for detection, and secondly reception means for receiving the radiation for detection as emitted by the chemical substance, said reception means being connected to the processor unit.

11. The detector system according to claim 4, in which the measurement unit comprises firstly emitter means for emitting excitation light towards the olfactory neuron to enable the excitation light to interact with the chemical substance to be detected in order to produce radiation for detection, and secondly reception means for receiving the radiation for detection as emitted by the chemical substance, said reception means being connected to the processor unit.

12. The detector system according to claim 5, in which the measurement unit comprises firstly emitter means for emitting excitation light towards the olfactory neuron to enable the excitation light to interact with the chemical substance to be detected in order to produce radiation for detection, and secondly reception means for receiving the radiation for detection as emitted by the chemical substance, said reception means being connected to the processor unit.
